SEQUENCE LISTING (18 sequences)

SEQ ID NO: 1 - protein, 8 aa, Artificial Sequence
  Synthetic polypeptide for a protein domain
CXMLNPPN

SEQ ID NO: 2 - protein, 8 aa, Artificial Sequence
  Synthetic polypeptide for a protein domain
CRMLNPPN

SEQ ID NO: 3 - protein, 8 aa, Artificial Sequence
  Synthetic polypeptide for a protein domain
CKMLNPPN

SEQ ID NO: 4 - protein, 8 aa, Artificial Sequence
  Synthetic polypeptide for a protein domain
CFMLNPPN

SEQ ID NO: 5 - protein, 8 aa, Artificial Sequence
  Synthetic polypeptide for a protein domain
CHMLNPPN

SEQ ID NO: 6 - protein, 8 aa, Artificial Sequence
  Synthetic polypeptide for a protein domain
CLMLNPPN

SEQ ID NO: 7 - protein, 8 aa, Artificial Sequence
  Synthetic polypeptide for a protein domain
CMMLNPPN

SEQ ID NO: 8 - protein, 8 aa, Artificial Sequence
  Synthetic polypeptide for a protein domain
CVMLNPPN

SEQ ID NO: 9 - protein, 8 aa, Artificial Sequence
  Synthetic polypeptide for a protein domain
CAMLNPPN

SEQ ID NO: 10 - protein, 8 aa, Artificial Sequence
  Synthetic polypeptide for a protein domain
CIMLNPPN

SEQ ID NO: 11 - DNA, 26 bp, Artificial Sequence
  primer
cggaattccagagctcccctgccttc

SEQ ID NO: 12 - DNA, 29 bp, Artificial Sequence
  primer
gctctagacatagagaaatgaatgcgttt

SEQ ID NO: 13 - DNA, 27 bp, Artificial Sequence
  primer
cggaattccagagtcactcctgccttc

SEQ ID NO: 14 - DNA, 29 bp, Artificial Sequence
  primer
gctctagacaaagagaaataggctcgttt

SEQ ID NO: 15 - DNA, 33 bp, Artificial Sequence
  primer
gaaattgggggggttaagcatgaaacattggcc

SEQ ID NO: 16 - DNA, 33 bp, Artificial Sequence
  primer
ggccaatgtttcatgcttaacccccccaatttc

SEQ ID NO: 17 - DNA, 32 bp, Artificial Sequence
  primer
gggggttaagcatcctacattggccataagtc

SEQ ID NO: 18 - DNA, 32 bp, Artificial Sequence
  primer
gacttatggccaatgtaggatgcttaaccccc

(19) United States
(12) Patent Application Publication
(10) Pub. No.: US 2008/0199472 A1
(43) Pub. Date: Aug. 21, 2008

(54) Use of a polypeptide domain to modulate the tumorigenic and metastatic potential of cancer cells

(75) Inventors: Hilde Revets, Meise (BE); Patrick De Baetselier, Antwerpen (BE); Nick Devoogdt, Brussel (BE); Gholamreza Hassanzadeh Ghassabeh, Watermael (BE)

Correspondence Address: TRASK BRITT, P.O. BOX 2550, SALT LAKE CITY, UT, 84110, US

(73) Assignees: Vlaams Interuniversitair Instituut Voor Biotechnologie VZW, Zwijnaarde (BE); Vrije Universiteit Brussel, Brussel (BE)

(21) Appl. No.: 12/070,239

(22) Filed: Feb. 14, 2008

Related U.S. Application Data
(62) Division of application No. 11/269,070, filed on Nov. 7, 2005.
Application No. 11/269,070 is a continuation of application No. PCT/EP2004/050627, filed on Apr. 28, 2004.

(30) Foreign Application Priority Data
Jul. 5, 2003 (EP) 03101265.1

(51) Int. Cl. A61K 39/395; A61K 38/00; C12N 5/06; A61P 43/00; C12Q 1/68

(52) U.S. Cl. 424/138.1; 514/16; 435/6; 435/375

(57) ABSTRACT

The present invention reates to the use of a polypeptide domain to modulate the tumorigenic and metastatic potential of cancer cells. More specifically, the present invention relates to a domain of a Secretory Leukocyte Protease Inhibitor (SLPI) to modulate tumor invasiveness and/or metastasis. It further relates to compounds, such as antibodies, that interact with the domain and repress the tumor invasiveness and/or the metastasis.

### CROSS-REFERENCE TO RELATED APPLICATION

This application is a divisional of U.S. patent application Ser. No. 11/269,070, filed Nov. 7, 2005, pending, which application is a continuation of International Application No. PCT/EP2004/050627, filed Apr. 28, 2004, published in English as PCT International Publication No. WO 2004/098626 on Nov. 18, 2004, which itself claims priority from EP 03101265.1 filed Jul. 5, 2003, the contents of each of which are incorporated by this reference.

### TECHNICAL FIELD

The present invention relates to the use of a polypeptide domain to modulate the tumorigenic and metastatic potential of cancer cells. More specifically, the present invention relates to a domain of a Secretory Leukocyte Protease Inhibitor (SLPI) to modulate tumor invasiveness and/or metastasis. It further relates to compounds, such as antibodies, that interact with the domain and repress the tumor invasiveness and/or the metastasis.

### BACKGROUND

Tumor progression is generally associated with extensive tissue remodeling to provide a proper environment for tumor growth, angiogenesis, and invasion and metastasis of cancer cells.⁽¹⁾ An impressive amount of data reveals that, among many factors, proteases expressed by cancer and/or stromal cells are key players in this process. Indeed, due to their ability to activate and release cytokines and growth factors and to degrade components of the extracellular matrix, proteases are necessary to provide optimal conditions for growth and invasion of cancer and endothelial cells. Expression of corresponding protease inhibitors in tumors is one way to control the activity of these enzymes. Protease inhibitors are therefore expected to be anti-malignant.⁽²⁾ However, serine protease inhibitors (SPIs) are often overexpressed in different tumor types,⁽³⁻⁷⁾ suggesting that overexpression of these inhibitors might favor tumor progression.⁽⁸⁾ Indeed, it has been demonstrated that overexpression of a number of SPIs from the serpin and kunitz families results in enhancement of cancer cell malignancy.⁽⁹⁻¹²⁾ None of the kazal-type SPIs has yet been shown to promote malignancy of cancer cells.

Secretory Leukocyte Protease Inhibitor (SLPI) is a member of the kazal-type SPI family. SLPI inhibits elastase, cathepsin G, trypsin and chymotrypsin⁽¹³⁾ and plays a significant role in protection against neutrophil proteases during massive inflammatory responses.⁽¹⁴⁻¹⁷⁾ The function of SLPI has been the subject of extensive investigation, since besides its function as an inhibitor of inflammatory proteases, SLPI exerts pleiotropic activities in different biological systems. For example, SLPI promotes wound healing⁽¹⁸⁾ and in vitro cell proliferation,^{(19, 20)} inhibits HIV infection⁽²¹⁾ and NF-κB activation,⁽²²⁾ lyses bacteria⁽²³⁾ and modulates macrophage functions.⁽²⁴⁾ Some of the activities of SLPI are independent of its protease inhibitory capacity towards certain proteases.⁽²¹⁻²⁴⁾

Several studies have reported a direct correlation between SLPI expression levels and tumor progression.^{(7, 25-28)} Moreover, WO9845431 discloses that SLPI has cancer metastasis potency, and that SLPI antisense RNA may be used for down-regulating the metastasis potency. WO9845431 further discloses a method for screening a compound having cancer metastasis inhibitory ability, comprising (a) contacting a test sample with the SLPI protein and (b) selecting compounds having the activity to bind the SLPI protein.

However, as mentioned above, it is known that SLPI protein can exert different functions, such as the inhibition of serine proteases, the activation of NF-κB, the modulation of the phenotype of macrophages, the inhibition of HIV infectivity of monocytes, and the induction of cancer metastasis potency. The different activities may be attributed to different domains in the protein.

Surprisingly, we found the role of SLPI in the malignant behavior of Lewis lung carcinoma 3LL-S cells can be attributed to a small specific domain in the protein. Even more surprisingly, we could demonstrate that this function of SLPI is dependent on its protease-inhibitory activity, but not on its ability to enhance cell proliferation. Moreover, unwanted SLPI overexpression is remarkably limited to the female reproductive organ, making SLPI and SLPI variants extremely useful for the diagnosis and treatment of ovarian cancers.

A first aspect of the invention is the use of a polypeptide comprising SEQ ID NO:1 to modulate tumor invasiveness and/or metastasis. Preferably, the tumor is an ovarian tumor. Preferably, the polypeptide is not SLPI. Preferably, the polypeptide is essentially consisting of SEQ ID NO:1, even more preferably, the sequence is consisting of SEQ ID NO:1. Preferably, the polypeptide comprises a SEQ ID NO:1 selected from the group consisting of SEQ ID NO:6 (human sequence), SEQ ID NO:7 (mouse sequence), SEQ ID NO:8, SEQ ID NO:9 and SEQ ID NO:10. Even more preferably, SEQ ID NO:1 is identical to SEQ ID NO:6. Preferably, the modulation is an inhibition of tumor invasiveness and/or metastasis. The domains are promoting tumor invasiveness and/or metastasis when placed in an SLPI context. However, as it is shown that the protease inhibitor domain binds to serine proteases such as elastase, and that the tumor promoting activity coincides with the protease-inhibitory activity, peptides and polypeptides comprising SEQ ID NO:1, but differing in sequence from SLPI protein for the other parts of the molecule may outcompete SLPI protein in binding the serine proteases without exerting the tumor promoting effect.

Another aspect of the invention is the use of a polypeptide comprising SEQ ID NO:2, SEQ ID NO:3, SEQ ID NO:4 or SEQ ID NO:5 to inhibit tumor invasiveness and/or metastasis. It has been shown indeed that mutant SLPI proteins comprising those domains have lost their tumor inducing capacity. Replacing, by gene therapy, of the tumor inducing form by the inactive mutant, would stop tumor development and metastasis.

A further aspect of the invention is the use of a compound, comprising SEQ ID NO:1, to isolate compounds that suppress tumor invasiveness and/or metastasis. Preferably, the tumor is an ovarian tumor. Preferably, the polypeptide is essentially consisting of SEQ ID NO:1, even more preferably, the sequence is consisting of SEQ ID NO:1. Preferably, the polypeptide comprises a SEQ ID NO:1 selected from the group consisting of SEQ ID NO:6, SEQ ID NO:7, SEQ ID NO:8, SEQ ID NO:9 and SEQ ID NO:10. Even more preferably, SEQ ID NO:1 is identical to SEQ ID NO:6. Indeed, as the SLPI protein interaction seems to be essential for the tumor inducing capacity, every compound that disturbs this interaction will have tumor reducing effect. Such compounds can be, as a non-limiting example, antibodies that bind on SEQ ID NO:1, or peptidomimetics of SEQ ID NO:1, that can outcompete the binding of SLPI protein with its substrate.

Methods to study protein-protein interaction are known to the person skilled in the art; the methods can be adapted to isolate compounds that destabilize the protein-protein interaction. As a non-limiting example, such methods have been described in WO03004643, WO9813502 and U.S. Pat. No. 5,733,726. To screen the compounds, SLPI protein can be used in combination with every possible SLPI substrate. As a non-limiting example, chymotrypsin, trypsin, cathepsin G or elastase can be used. Preferably, SLPI protein together with elastase is used to screen or compounds that disrupt the protein-protein interaction.

Still another aspect of the invention is the use of a compound, which is decreasing the inhibiting activity of SLPI to a serine protease to suppress tumor invasiveness and/or metastasis. Preferably, the tumor is an ovarian tumor. Preferably, the SLPI is human SLPI and the serine protease is elastase. Preferably, the compound is an antibody binding SEQ ID NO:1.

### DEFINITIONS

The following definitions are set forth to illustrate and define the meaning and scope of various terms used to describe the invention herein.

Bind(ing) means any interaction, be it direct or indirect. A direct interaction implies a contract between the binding partners. An indirect interaction means any interaction whereby the interaction partners interact in a complex of more than two compounds. The interaction can be completely indirect, with the help of one or more bridging molecules, or partly indirect, where there is still a direct contact between the partners, which is stabilized by the additional interaction of one or more compounds.

Compound means any chemical of biological compound, including simple or complex organic and inorganic molecules, peptides, peptido-mimetics, proteins, antibodies, carbohydrates, nucleic acids or derivatives thereof.

The terms protein and polypeptide as used in this application are interchangeable. Polypeptide refers to a polymer of amino acids and does not refer to a specific length of the molecule. This term also includes post-translational modifications of the polypeptide, such as glycosylation, phosphorylation and acetylation.

### BRIEF DESCRIPTION OF THE DRAWINGS

FIG. 1: Malignant potential of 3LL-S and 3LL-S-sc cells. (a) s.c. growth of 3LL-S and 3LLS-sc cells in C57B1/6 mice (P=0.0056 at 22 d.p.i.) (b) Lung-colonizing potential of 3LL-S and 3LL-S-sc cells in C57B1/6 mice at 32 d.p.i. (P=0.013 and 0.0081 for lung weight and number of lung nodules, respectively) (c) s.c. growth of 3LL-S and 3LL-S-sc cells in SCID mice (P=0.032 at 29 d.p.i.) (d) Lung-colonizing potential of 3LL-S and 3LL-S-sc cells in SCID mice at 21 d.p.i. (P=0.016 and 0.0020 for lung weight and number of lung nodules, respectively).

FIG. 2: mSLPI expression in 3LL-S and 3LL-S-sc cells (a) Northern blot analysis of expression of mSLPI and GAPDH. (b) Normalized mSLPI mRNA levels. The relative quantities of mSLPI mRNA were determined by densitometry and normalized using GAPDH.

FIG. 3: mSLPI overexpression enhances the malignancy of 3LL-S cells (a) Normalized mSLPI mRNA levels in the mock-transfectant NA1 and mSLPI-transfectant mD7. The relative quantities of mSLPI mRNA were determined by densitometry and normalized using GAPDH (b) s.c. growth of NA1 and mD7 in SCID mice (P=0.0011 at 27 d.p.i.) (c) Lung colonizing potential of NA1 and mD7 in SCID mice at 36 d.p.i. (P=0.023 and 0.014 for lung weight and number of lung nodules, respectively).

FIG. 4: The pro-malignant effect of hSLPI is dependent on its protease inhibitory activity (a) secretion levels of hSLPI, F- or R-hSLPI by A549, 3LL-S and 3LL-S-sc cells, 3LL-S mock-transfectant NA1, mSLPI-transfectant mD7, hSLPI-transfectants h2C5 and h4E5, F-hSLPI-transfectant F-h1A8 and R-hSLPI-transfectant R-h2D8 (b) s.c. growth of NA1, h2C5, h4E5, F-h1A8 and R-h2D8 in SCID mice (P=0.0003 and 0.0001 for h2C5 and h4E5, respectively, as compared to NA1. P=0.0063 and 0.0012 for F-h1A8and R-h2D8, respectively, as compared to h4E5). P values were calculated from the data at 27 d.p.i. (c) Lung-colonizing potential of NA1, h2C5, h4E5, F-h2A8 and R-h2D8 in SCID mice at 36 d.p.i. (lung weight: P<0.0001 for h2C5 and h4E5, as compared to NA1. P=0.19 and 0.0007 for F-h1A8 and R-h2D8, respectively, as compared to h4E5. Number of lung nodules: P<0.0001 for h2C5 and h4E5, as compared to NA1. P=0.0054 and 0.0012 for F-h1A8 and Rh2D8, respectively, as compared to h4E5).

FIG. 5: Effect of SLPI expression on the in vitro cell proliferation of 3LL-S cells. Cell proliferation rates of transfected 3LL-S cells were measured by [3H]-thymidine uptake. The data shown are representative of five independent experiments. P<0.0001 for mD7, h2C5, h4E5 and R-h2D8 and P=0.4922 for F-h1A8, as compared to NA1. P<0.0001 for F-h1A8 and P=0.8381 for R-h2D8, as compared to h4E5.

FIG. 6: Specific expression of SLPI in the female reproductive organ. Normalized SLPI expression in tumor tissue (T) versus normal tissue (N) in cancers of the breast (n=50), female reproductive organ (n=57), intestinal tract (n=55), stomach (n=27), lung (n=21), kidney (n=20), thyroid (n=6), prostate (n=4) and pancreas (n=1), using cDNA dot blot hybridizations. Intensity difference, ratio and score were calculated for each individual patient. Results are presented as mean±95% CI.

### EXAMPLES

### Materials and Methods to the Examples

Mice. −8 weeks old female C57B1/6 (Harlan, The Netherlands) and CB17/IcrHanHsd-SCID mice (Harlan, The Netherlands) were used in all experiments.

Cell lines and culture conditions. The 3LL-S cell line has been described elsewhere.⁽²⁹⁾ The 3LL-S-sc cell line was obtained by s.c. inoculation of 2×10⁶ 3LL-S cells in C57B1/6 mice, followed by removal and homogenization of the resulting tumor tissue and in vitro propagation of cancer cells for at least 10 days to eliminate contaminating host cells. The human lung carcinoma cell line A549 was kindly provided by Dr. M. Mareel (RUG, Ghent, Belgium). All cell lines were maintained in RPMI 1640 supplemented with 0.3 mg/ml L-glutamine, 100 units/ml penicillin, 0.1 mg/ml streptomycin, and 10% heat-inactivated fetal calf serum (Gibco BRL). Cells were grown in a humidified incubator at 37° C., containing 5% CO2.

General molecular techniques. Unless otherwise noted, nucleic acids were handled according to standard protocols. PCR products were purified using the PCR Purification Kit (Qiagen) as recommended by the manufacturer. Nucleotide sequences were determined by the dideoxynucleotide chain termination method. Nucleic acid homology searches were preformed using the FastA program. Total RNA and mRNA were prepared using Trizol reagent (Gibco BRL) and Fasttrack 2.0 Kit (Invitrogen), respectively, following the suppliers' recommendations.

Construction and screening of a subtracted cDNA library. A subtracted cDNA repertoire enriched for cDNA fragments up-regulated in 3LL-S-sc, as compared to 3LL-S cells, was generated using the PCR-Select cDNA Subtraction Kit (Clonetech), as instructed by the manufacturers. The cDNAs obtained from 3LL-S and 3LL-S-sc cells were used as driver and tester, respectively. The subtracted cDNA repertoire was cloned into the T/A cloning vector pCR2.1 (Invitrogen) and transformed into E. coli strain TOP10F′ (Invitrogen). Differential expression of cloned cDNA fragments was tested by northern blot using standard protocols. Probes were generated by PCR amplification of cDNA inserts and labeled using the Rediprime II random prime labeling system (Amersham Pharmacia Biotech). The membranes were exposed to a phosphor-imaging screen and developed using the Molecular Imager system (Biorad). The specific signals were quantified using the Molecular Analyst software (Biorad). The signals were normalized using the house-keeping gene GAPDH.

Tumorigenicity. 2×10⁶ cells were injected s.c. in the flank, and tumor length (L) and width (W) were measured at different time points using a caliper. The tumor volume (V) was calculated as V=W×W×L×0.4.

Evaluation of experimental metastatic potential. 2×10⁶ cells were injected i.v. via the tail vein. Lung-colonizing potential was measured by monitoring the lung weight and number of visible metastatic nodules after fixation in Bouin's solution (Sigma).

Transfection of 3LL-S cells with mSLPI, hSLPI, F-hSLPI or R-hSLPI. Using primers 5′-CGGAATTCCAGAGCTCCCCTGCCTTC-3′ (SEQ ID NO:11) and 5′-GCTCTAGACATAGAGAAATGAATGCGTTT-3′ (SEQ ID NO:12), the full-length mSLPI cDNA (including the signal peptide and the 3′ untranslated region) was obtained by RT-PCR on mRNA from 3LL-S cells. The full-length hSLPI cDNA was obtained by RT-PCR on total RNA from A549 cells using primers 5′-CGGAATTCCAGAGTCACTCCTGCCTTC-3′ (SEQ ID NO:13) and 5′-GCTCTAGACAAAGAGAAATAGGCTCGTTT-3′ (SEQ ID NO:14). Using primer pairs 5′-GAAATTGGGGGGGTTAAGCATGAAACATTGGCC-3′ (SEQ ID NO:15) and 5′-GGCCAATGTTTCATGCTTAACCCCCCCAATTTC-3′ (SEQ ID NO:16), or 5′-GGGGGTTAAGCATCCTACATTGGCCATAAGTC-3′ (SEQ ID NO:17) and 5′-GACTTATGGCCAATGTAGGATGCTTAACCCCC-3′ (SEQ ID NO:18), the codon for Leu72 of the mature hSLPI protein was mutated via PCR into a codon for Phe (F-hSLPI) or Arg (R-hSLPI), respectively (the nucleotides replacements are shown in bold). PCR products were cloned into the EcoRI/Xbal sites of the pcDNA3.1 (+)/Neo plasmid (Invitrogen). After sequence verification, the recombinant plasmids containing mSLPI, hSLPI, F-hSLPI or R-hSLPI cDNA, in parallel with the empty plasmid, were electroporated into 3LL-S cells following standard protocols. Subcloning and selection in the presence of neomycin (Gibco BRL) resulted in the isolation of stable transfectants. mSLPI expression in transfectants was evaluated by northern blot. Each northern blot was repeated three times. hSLPI, F-hSLPI or R-hSLPI secretion was evaluated using the “human SLPI ELISA Test Kit” (HyCult biotechnology). Three independent ELISAs were performed.

In vitro cell proliferation assay. Exponentially growing cancer cells were collected, thoroughly washed in RPMI and incubated for 24 hours in serum-free medium to synchronize the cells. The cells were collected, resuspended in serum-containing medium and seeded for 24 hours in six-fold at 10⁴ cells per well in 96-well plates. Cell proliferation was quantified in an 18-hour [³H]-thymidine incorporation assay.

Statistical analysis. Statistical analyses were performed by the two-tailed unpaired t-test.

### Example 1

### Subcutaneous Growth of 3LL-S Cells Enhances their Malignancy

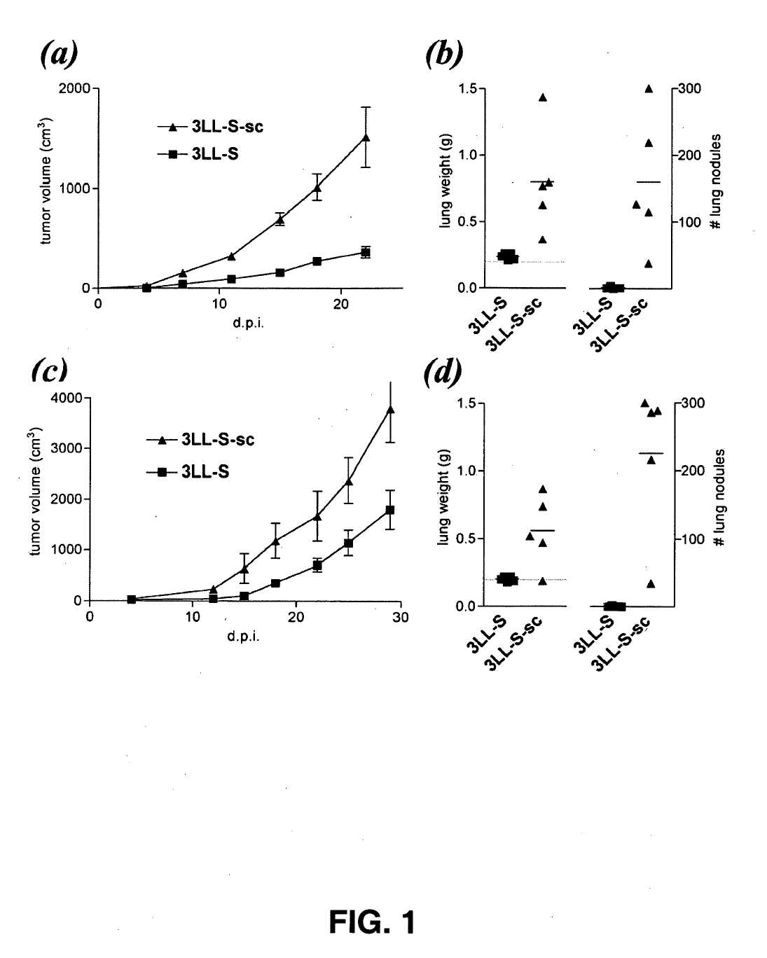

The 3LL-S cell line is a low-malignant subclone derived from the parental Lewis Lung Carcinoma.⁽²⁹⁾ The low-malignancy of these cells is reflected by their low tumorigenicity upon s.c. inoculation (FIGS. 1a and 1c) and low lung-colonizing potential after i.v. injection (FIGS. 1b and 1d), in both syngeneic C57B1/6 (FIGS. 1a and 1b) and immunodeficient SCID mice (FIGS. 1c and 1d). Upon s.c. growth in syngeneic C57B1/6 mice, 3LL-S cells become more malignant. Indeed, as compared to the parental 3LL-S cells, cancer cells derived from s.c. 3LL-S tumors (hereafter referred to as 3LL-S-sc cells) grow significantly faster in the flank of mice (FIGS. 1a and 1c). In addition, 3LL-S-sc cells colonize the lung more extensively than 3LL-S cells after i.v. injection (FIGS. 1b and 1d). These data show that 3LL-S-sc cells are significantly more malignant than 3LL-S cells, as manifested by their increased capacity to grow at a local site and to colonize the lung.

### Example 2

### Mouse SLPI Expression is Up-Regulated During s.c. Growth of 3LL-S Cells

In order to identify genes whose expression is modulated during s.c. growth of 3LL-S cells, the SSH approach was adopted. This approach led to the identification of a 480-bp cDNA fragment corresponding to the 3′ fragment of the mouse SLPI (mSLPI) mRNA.⁽¹³⁾

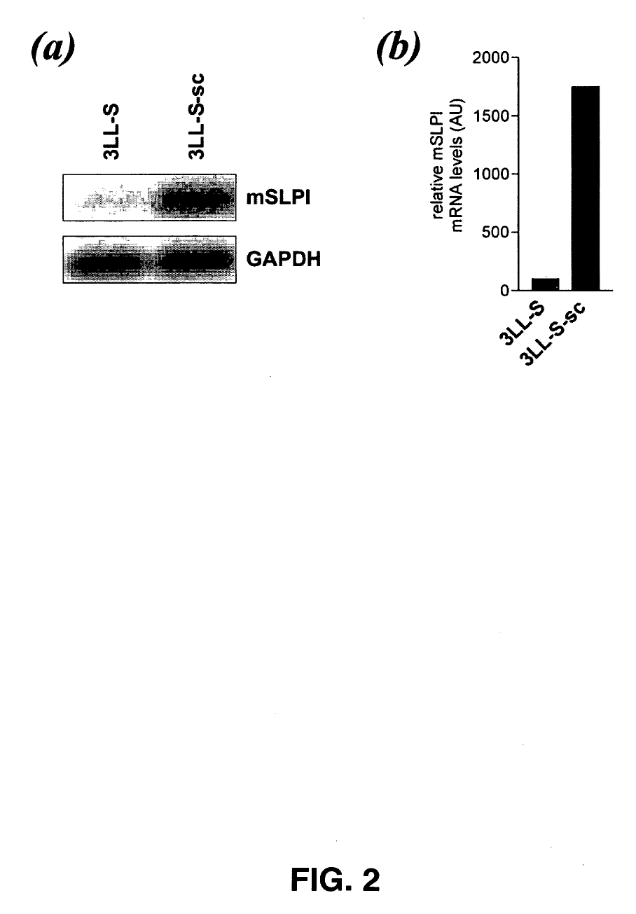

The up-regulation of mSLPI expression upon s.c. growth of 3LL-S cells was further validated by northern blot. These northern blot experiments (FIG. 2a) and subsequent normalization with the house-keeping gene GAPDH, revealed that the mSLPI mRNA level was about 15-fold higher in 3LL-S-sc cells as compared to 3LL-S cells (FIG. 2b).

### Example 3

### Mouse SLPI Overexpression Enhances the Malignancy of 3LL-S Cells

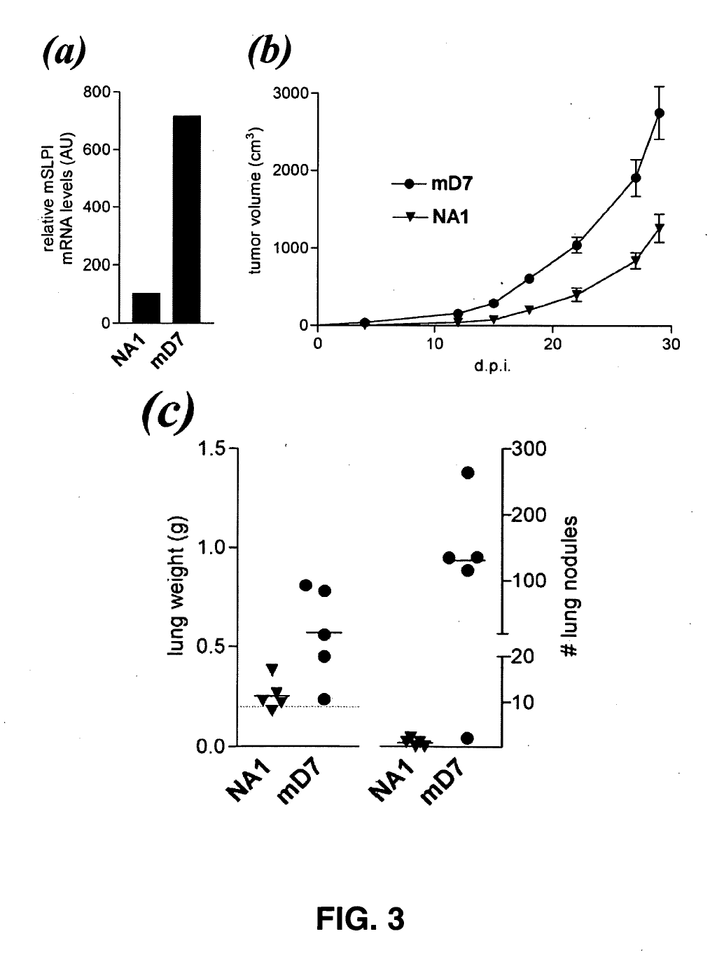

The above experiments revealed a direct correlation between mSLPI expression levels and the malignant behavior of 3LL-S and 3LL-S-sc cells. We next investigated whether elevated levels of mSLPI expression enhanced the tumorigenicity and/or lung-colonizing potential of 3LL-S cells. To this end, 3LL-S cells were transfected with a plasmid expressing mSLPI. As negative control-transfectant, the empty plasmid was introduced into 3LL-S cells. The stable mSLPI-transfectant mD7, in which the mSLPI mRNA level was about 7-fold higher than that in 3LL-S cells, was selected for further analysis (FIG. 3a). The control transfectant clone NA1, with mSLPI mRNA levels similar to that of 3LL-S, was used as negative control.

The role of mSLPI in increasing malignancy of 3LL-S cells was tested by measuring the tumorigenicity and lung-colonizing potential of the mSLPI overexpressing clone mD7 and the control mock-transfectant clone NA1. As shown in FIG. 3, a 7-fold mSLPI overexpression significantly enhanced tumor growth (FIG. 3b) and lung-colonizing potential (FIG. 3c) of 3LL-S cells injected s.c. or i.v., respectively.

### Example 4

### Human SLPI (hSLPI) Expression in 3LL-S Cells Enhances Their Malignancy

Although mSLPI and hSLPI exhibit only 58% identity at the amino acid level, the proteases they inhibit are similar.⁽³⁰⁾ Besides, it has been shown that, similar to mSLPI, hSLPI is up-regulated during cancer progression.^{(25, 27)} Hence, we investigated whether, similar to mSLPI, hSLPI also promotes the malignancy of 3LL-S cells.

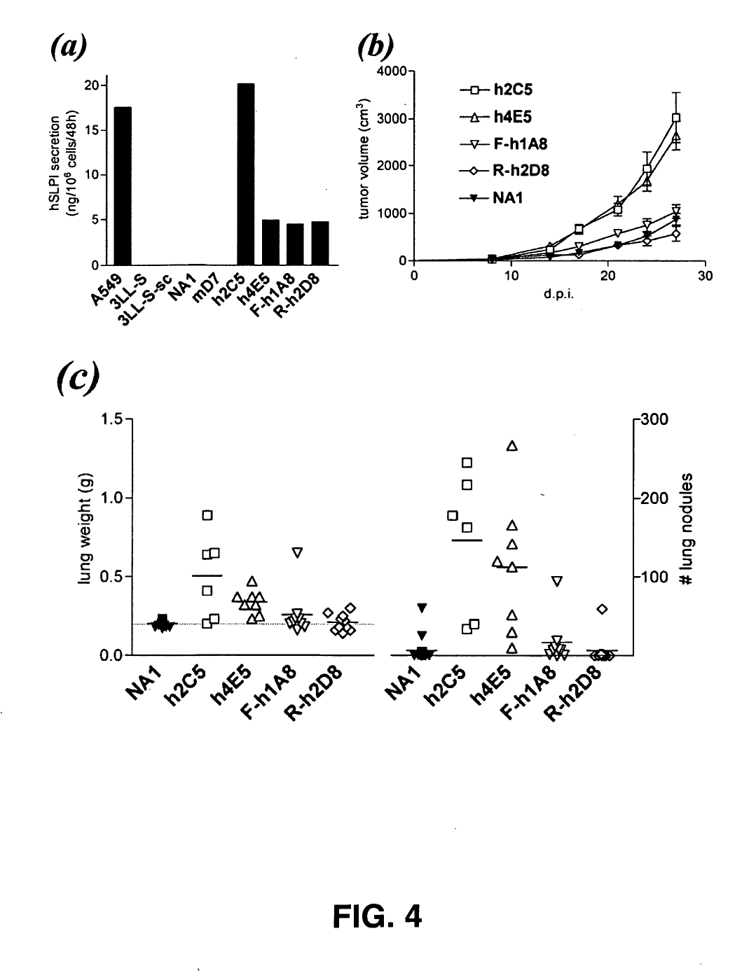

To assess the malignancy-promoting activity of hSLPI, 3LL-S cells were transfected with a plasmid expressing hSLPI. Based on ELISA, two stable hSLPI-transfectants, clones h2C5 and h4E5, secreting about 20 and 5 ng hSLPI per 10⁶ cells per 48 h, respectively, were selected for further analysis. Conditioned medium from the human lung cancer cell line A549 was used as positive control in ELISA. 3LL-S, 3LL-S-sc cells, mSLPI-transfectant clone mD7 and the control-transfectant clone NA1 did not yield any ELISA signal in these experiments, demonstrating that ELISA signals obtained with hSLPI-transfectants were specific for hSLPI (FIG. 4a).

The effect of hSLPI on the malignancy of 3LL-S cells was then tested by measuring the tumorigenicity and lung-colonizing potential of the hSLPI-expressing clones h2C5 and h4E5 and the control mock-transfectant clone NA1. Similar to the mSLPI-transfectant mD7, the hSLPI-transfectants h2C5 and h4E5 grew much faster in the flank of mice than the mocktransfectant NA1 (FIG. 4b). As measured by both the number of lung nodules and lung weight, both hSLPI-transfectants exhibited a significantly higher lung-colonizing potential as compared to the mock-transfectant clone NA1 (FIG. 4c). A hSLPI secretion level of about 5 ng per 10⁶ cells per 48 h was sufficient to enhance the malignancy of 3LL-S cells; indeed, although clones h2C5 and h4E5 differed about 4-fold in their hSLPI secretion levels, they did not differ significantly in their tumorigenicity (P=0.52) and lung-colonizing potential (P=0.12 and 0.48 for lung weight and number of lung nodules, respectively). Therefore, despite the differences in their amino acid sequences, both mouse and human SLPIs enhance the malignant potential of 3LL-S cells.

### Example 5

### The Protease Inhibitory Activity of hSLPI is Involved in its Malignancy-Promoting Capacity

To assess the role of the protease-inhibitory activity of hSLPI in its capacity to promote malignancy of 3LL-S cells, two mutant hSLPIs were generated. In these mutants, Leu72 of the mature WT hSLPI protein was replaced by Phe (in F-hSLPI) or Arg (in R-hSLPI). These mutations have already been shown to result in a drastic alteration in the inhibitory activity of hSLPI towards serine proteases.⁽³¹⁾

Plasmids expressing each of these mutants were used to transfect 3LL-S cells. Two transgenic cell lines, F-hSLPI-transfectant F-h1A8 and R-hSLPI-transfectant R-h2D8, having expression levels similar to that of the WT hSLPI-transfectant h4E5, were selected for further study (FIG. 4a). The transfectants F-h1A8 and R-h2D8 were compared to the mock transfectant NA1 and the hSLPI-transfectant h4E5 for their capacity to colonize the lung and to grow locally. As depicted in FIG. 4b, both mutant hSLPI-transfectants grew significantly slower than the hSLPI-transfectant h4E5 and exhibited growth curves similar to the mock transfectant NA1. Moreover, after i.v. injection, both mutant hSLPI-transfectants F-h1A8 and R-h2D8 colonized the lung less efficiently than the WT hSLPI-transfectant h4E5. This was reflected by both a decreased number of lung nodules and a lower lung weight, the latter to a lesser extent (FIG. 4c). These experiments demonstrate that the protease inhibitory activity of hSLPI is involved in the promotion of 3LL-S malignancy.

### Example 6

### The Pro-Malignant Activity of SLPI is not Mediated by its Effect on In vitro Cell Proliferation

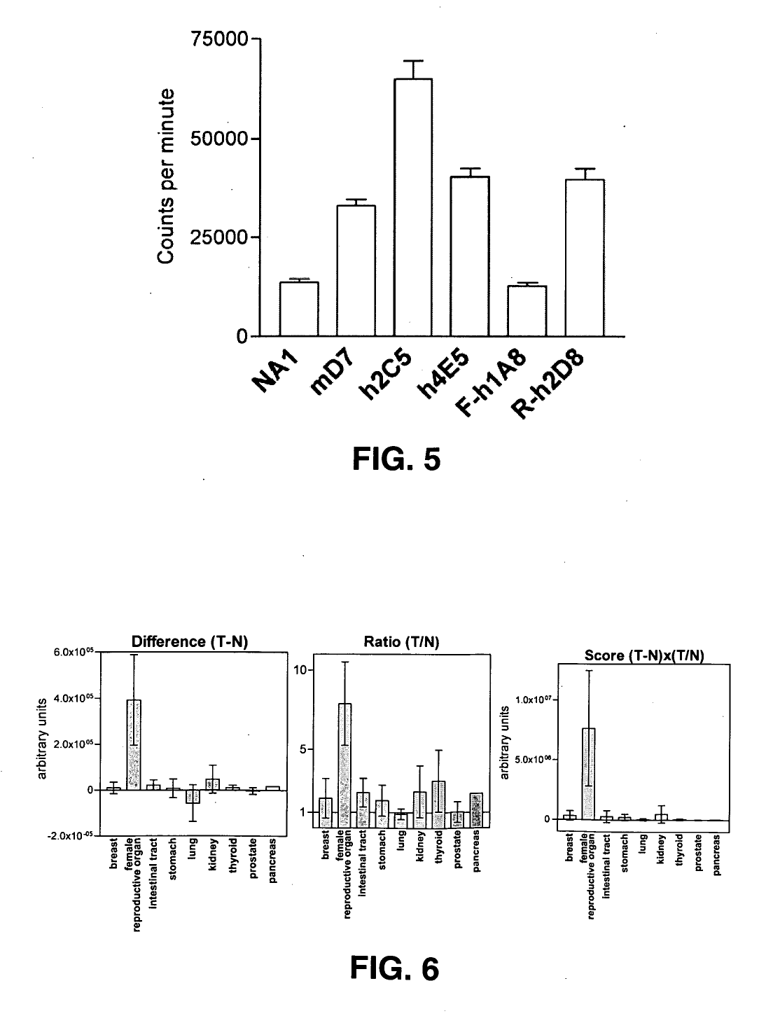

In view of two recent reports linking SLPI expression with in vitro proliferation rates of human endometrial cells,^{(19, 20)} the influence of SLPI on 3LL-S cell proliferation was tested. To this end, in vitro proliferation rates of mock- and SLPI-transfectants were compared. SLPI-transfectant clones mD7, h2C5 and h4E5 proliferated, respectively, 2.4, 4.8 and 3.0 times faster than the mock-transfectant NA1, demonstrating that SLPI indeed promotes the proliferation of 3LL-S cells in vitro (FIG. 5). When Leu72 was mutated to Phe, the in vitro growth-stimulating effect of hSLPI was abrogated: transfectant F-h1A8 proliferated significantly slower than the hSLPI-transfectant h4E5 and exhibited the same proliferation rate as the mock-transfectant NA1. However, replacement of Leu72 by Arg did not change the effect of hSLPI on the proliferation of 3LL-S cells; indeed, transfectant R-h2D8 proliferated as fast as the hSLPI-transfectant h4E5 and significantly faster than the mock-transfectant NA1 (FIG. 5). Taken together these data and the in vivo malignancy of these cells, there is not always a direct correlation between the in vitro proliferation rate of these cells and their in vivo malignant behavior. Therefore, the pro-malignant activity of SLPI cannot be explained solely by its effect on in vitro cell proliferation.

### Example 7

### SLPI Possible Marker for Gynecological Cancers

SLPI and HE4, two members of the WAP-family of small acidic proteins that share the same four-disulfide core domain structure, have been reported to be overexpressed in gynecological tumor tissue.⁽⁷⁾ These gene inductions are probably due to gene amplifications since the chromosomal region containing the WAP-proteins is frequently amplified in gynecological cancers.⁽³⁸⁾ HE4, although its function is unknown, has recently been proposed to be a new biomarker for ovarian carcinoma.⁽³⁹⁾ As shown in this invention, SLPI has tumor-promoting properties in an artificial mouse tumor model.

To determine whether SLPI might also serve as a marker for gynecological cancers, tumor-specific expression was monitored in patients with cancers originating from a wide array of organs, including the female reproductive tract. Therefore, nylon membranes spotted with cDNAs derived from normal (N) and tumor tissue (T) isolated from individual patients (Cancer Profiling Array, BD Biosciences, Palo Alto, Calif.) were hybridized with a SLPI-specific probe, signals were quantified and normalized against the house-keeping gene ubiquitin. Among all organs tested, only in cancers of the female reproductive organ tumor-specific SLPI-up-regulation was evident, as measured by the criteria “difference” (T−N), “ratio” (T/N) and “score” (T−N)×(T/N) (see figure). Here, expression was in 79% of the cases higher in tumor than in normal tissue, and this up-regulation was not confined to specific histological grade, stage or cell type. Also, only in cancers of the female reproductive tract, but not in other types, SLPI-up-regulation was statistically significant (P=0.0002) as judged by the Paired two-tailed t test.

Altogether, these data clearly indicate that in gynecological cancers, but not in other cancer types tested here, SLPI expression is not only frequently up-regulated, but also that the differences in SLPI expression between tumor and normal tissues are significantly high. Therefore, in addition to HE4, SLPI might be used as a new marker for gynecological cancer.

SEQUENCE LISTING

SEQ
ID
No

| | |
|---|---|
| 1 | CXMLNPPN |
| 2 | CRMLNPPN |
| 3 | CKMLNPPN |
| 4 | CFMLNPPN |
| 5 | CHMLNPPN |
| 6 | CLMLNPPN |
| 7 | CMMLNPPN |
| 8 | CVMLNPPN |
| 9 | CAMLNPPN |
| 10 | CIMLNPPN |
| 11 | cggaattcca gagctcccct gccttc |
| 12 | gctctagaca tagagaaatg aatgcgttt |
| 13 | cggaattcca gagtcactcc tgccttc |
| 14 | gctctagaca aagagaaata ggctcgttt |
| 15 | gaaattgggg gggttaagca tgaaacattg gcc |
| 16 | ggccaatgtt tcatgcttaa cccccccaat ttc |
| 17 | gggggttaag catcctacat tggccataag tc |
| 18 | gacttatggc caatgtagga tgcttaaccc cc |

1. A method of modulating tumor invasiveness and/or metastasis in a subject suffering from a tumor comprising administering a polypeptide comprising SEQ ID NO:1.

2. The method of claim 1, wherein said polypeptide comprises SEQ ID NO:6, SEQ ID NO:7, SEQ ID NO:8, SEQ ID NO:9 or SEQ ID NO:10.

3. The method of claim 1, wherein said modulation is an inhibition of tumor invasiveness and/or metastasis.

4. The method of claim 3, wherein said polypeptide comprises SEQ ID NO:2, SEQ ID NO:3, SEQ ID NO:4 or SEQ ID NO:5.

5. A method of identifying compounds that suppress tumor invasiveness and/or metastasis comprising
introducing into a host cell a nucleotide sequence comprising a DNA sequence encoding the polypeptide of SEQ ID NO:1 and a gene comprising a DNA sequence encoding a substrate of SEQ ID NO:1;
introducing into the host cell a test molecule polypeptide to be tested for its capability to disrupt an interaction between SEQ ID NO:1 and the substrate of SEQ ID NO:1; and
subjecting the host cell to conditions
(i) allowing said SEQ ID NO:1 and the substrate of SEQ ID NO:1 to be expressed in quantities sufficient to allow specific interaction between SEQ ID NO:1 and the substrate of SEQ ID NO:1 such that when transcription is activated, a toxin is expressed and said host cell dies if said test molecule is not capable of disrupting said protein—protein interaction, and
(ii) allowing said test molecule polypeptide, if it is capable of doing so, to disrupt said interaction between said SEQ ID NO:1 and the substrate of SEQ ID NO:1, thereby disrupting transcriptional activation of said detectable nucleotide sequence, which disrupts expression of said toxin reporter gene, and results in survival of the host cell.

6. The method of claim 5, wherein said test molecule polypeptide comprises SEQ ID NO:6, SEQ ID NO:7, SEQ ID NO:8, SEQ ID NO:9 or SEQ ID NO:10.

7. The method of claim 1, wherein said tumor is an ovarian tumor.

8. A method for decreasing the inhibiting activity of Secretory Leukocyte Protease Inhibitor to a serine protease to suppress tumor invasiveness and/or metastasis comprising administering to a subject suffering from a tumor a compound binding SEQ ID NO:1.

9. The method of claim 8, wherein said serine protease is elastase.

10. The method of claim 8, wherein, said Secretory Leukocyte Protease Inhibitor is human Secretory Leukocyte Protease Inhibitor.

11. The method of claim 8, wherein said compound is an antibody.

12. The method of claim 8, wherein said tumor is an ovarian tumor.

13. The method of claim 2, wherein said modulation is an inhibition of tumor invasiveness and/or metastasis.

14. A method for decreasing the activity of Secretory Leukocyte Protease Inhibitor (SLPI) to promote invasiveness and/or metastasis of a cell, the method comprising administering to said cell a compound specifically binding SLPI.

15. The method of claim 14, wherein, said Secretory Leukocyte Protease Inhibitor is human Secretory Leukocyte Protease Inhibitor.

16. The method of claim 14, wherein said compound is an antibody.

17. The method of claim 14, wherein said cell is an ovarian cell.

18. A method of decreasing the colonizing activity of a cell, the method comprising interfering with Leu72 of SLPI in said cell.

19. The method according to claim 18, wherein interfering with Leu72 of SLPI in said cell comprises mutating Leu72.

20. A method of modulating the invasiveness of a cell comprising administering to the cell a polypeptide comprising SEQ ID NO:1.

21. The method according to claim 20, wherein said polypeptide comprises SEQ ID NO:6, SEQ ID NO:7, SEQ ID NO:8, SEQ ID NO:9 or SEQ ID NO:10.

22. The according to claim 20, wherein said modulation is an inhibition of the invasiveness of the cell.

23. The method of claim 22, wherein said polypeptide comprises SEQ ID NO:2, SEQ ID NO:3, SEQ ID NO:4 or SEQ ID NO:5.
